(19) United States
(12) Patent Application Publication
(10) Pub. No.: US 2006/0263304 A1
(43) Pub. Date: Nov. 23, 2006

(54) Method of controlling alcohol intake and reversing alcohol effects

(76) Inventor: James A. Halikas, Naples, FL (US)

Correspondence Address: James A. Halikas, M.D., 783 Tramore Lane, Naples, FL, 34108, US

(21) Appl. No.: 11/134,675

(22) Filed: May 23, 2005

(51) Int. Cl. A61L 9/04

(52) U.S. Cl. 424/45

(57) ABSTRACT

A method for controlling or reversing the alcohol intake in the non-alcohol dependent individual both before or during their alcohol imbibing. This is accomplished by self-administering a spray directed into sensitive mucosal vein endings of the individual. There are at least three technologies to accomplish the above noted task. The first one is a nasal spray which is directed into the nasal passage. The second spray is directed into the mouth of the individual so that the medication can be absorbed by the buccal mucosal membrane in the mouth of the individual. The third medicated spray technology is pulmonary inhaler aerosolization which is absorbed in the tissues of the lung. All of the above noted sprays by-pass the liver and the intestines. The medication, Flumazenil, is the medication used in any of these sprays to accomplish the desired effect.

### FIELD OF THE INVENTION

The field of this invention involves the use of alcohol. The use of alcohol is a widespread phenomenon that can lead to dire consequences in the lives of many individuals. The consumption of alcohol, when used in moderation, is a socially accepted fact. This invention moderates the use and effects of alcohol acutely in the non-alcohol dependent individual.

### BACKGROUND OF THE INVENTION

The inventive method relates to the use of alcohol in the general population directed to either social drinkers or any individual who may want to control any alcohol intake during certain occasions. There are two co-pending applications, the first one is application Ser. No. 11/049,067 and the second one is a CIP of the first one. Both prior applications are directed to a “Method of preventing a relapse in the abstinent substance dependent individual” These applications involve the consumption of alcohol and other substances in a substance dependent individual where the consumption has progressed to abuse of the above noted substances and finally progressed to a state of illness.

The criteria for the diagnosis of alcohol dependence have achieved general consensus in the medical field in the last 20 years. There is a clear separation between someone who can receive a diagnosis of alcohol dependence and someone who cannot receive such a diagnosis. There are two texts that are determinative and concordant. The American Psychiatric Association Diagnostic and Statistical Manual 4th Edition, and in the international arena, the International Classification of Diseases, 9th Revision, Clinical Modification.

Alcohol dependence is subsumed under the more general Diagnosis of Substance Dependence, with the specifier that the substance is alcohol. The descriptive criteria are extensive and require a long term pattern of mal adaptive use of alcohol, with several specific medical and behavioral adverse consequences. The complete diagnostic criteria, are presented in the APA, DSM IV., and are here reproduced

Substance Dependence

A maladaptive pattern of substance use, leading to clinically significant impairment or distress, as manifested by three (or more) of the following, occurring at any time in the same 12-month period:
(1) Tolerance, as defined by either of the following:
   (a) a need for markedly increased amounts of the substance to achieve intoxication or desired effect;
   (b) markedly diminished effect with continued use of the same amount of the substance;
(2) Withdrawal, as manifested by either of the following:
   (a) the characteristic withdrawal syndrome for the substance (refer to criteria A and B of the criteria sets for Withdrawal from the specific substances);
   (b) the same (or closely related) substance is taken to relieve or avoid withdrawal symptoms.
(3) the substance is often taken in larger amounts or over a longer period than was intended.
(4) there is a persistent desire or unsuccessful efforts to cut down or control substance use.
(5) a great deal of time is spent in activities necessary to obtain the substance (e.g., visiting multiple doctors or driving long distances), use the substance (e.g., chain-smoking), or recover from its effect.
(6) important social, occupational, or recreational activities are given up or reduced because of substance use.
(7) the substance use is continued despite knowledge of having a persistent or recurrent physical or psychological problem that is likely to have been caused or exacerbated by the substance (e.g., current cocaine-induced depression, or continued drinking, despite recognition that an ulcer was made worse by alcohol consumption).

Thus, the inventive concept of this application is directed to an entirely different population, that is, the normal social drinker.

### BRIEF DESCRIPTION OF THE INVENTION

The inventive concept of this invention involves the use of the medication “Flumazenil” in a spray or inhalation delivery system to control, deter or prevent alcohol consumption during a particular occasion of potential alcohol use, but in this totally different population from those afflicted with the disease of alcohol dependence. In this invention the medication “Flumazenil” may be used in any spray delivery system that provides direct absorption through a mucous membrane into the blood.

Flumazenil has been in the medical literature for at least 15 to 20 years. It is a Benzodiazepine antagonist, a receptor blocker that blocks the action of Librium, Valium, Xanax, Ativan or other Benzodiazepines.

These medications are addictive and create a dependency in themselves. It turns out that there is a receptor in the brain for these molecules, the Benzodiazepine receptor.

Flumazenil blocks the Benzodiazepine receptor, or in the alternative, if one has taken one of these medications (one of the Benzodiazepines) and if Flumazenil is given to a patient, it will push out these molecules and will place the patient into an instant withdrawal state. Thus, Flumazenil is routinely used in the emergency room setting when people have overdosed and almost killed themselves.

Flumazenil is also used to reverse the effects of Benzodiazepines (e.g. Diazepine and temazepam) which are often used to induce sedation prior to minor outpatient surgical procedures.

The Benzodiazepines work by acting on receptors in the brain (GABA receptors) causing the release of a chemical called GABA (gamma amino butyric acid). GABA is a major inhibitory chemical neuro transmitter in the brain involved in, for example, sleep induction and control of trembling. Benzodiazepines act by increasing the activity of GABA, thereby reducing the functioning of certain areas in the brain. This results in sleepiness, a decrease in shaking and a relaxation of muscles.

Flumazenil reverses the effects of the Benzodiazepines by competing with them for the GABA receptors. Flumazenil binds to the receptors, preventing Benzodiazepines from acting on them. This blocks their effect and causes sedation to be reversed.

Here, the inventive concept uses this medication (Flumazenil) for a new use, in surprising new methodologies, convenient spray delivery devices or technologies, never before used for administering this medication. This inventive concept uses this medication “Flumazenil” for moderating alcohol consumption and/or control never before dreamed of, under the unheard of control of the patient with excellent clinical results. This aid in the control or reversal of alcohol consumption, through the use of Flumanezil via a novel route of administration is remarkable and a genuinely novel invention.

### DETAILED DESCRIPTION OF THE INVENTION

As mentioned above, the inventive concept is directed to a method of controlling alcohol intake and reversing the effects of alcohol acutely in a non-alcohol dependent individual. The non-alcoholic individual can be described as a social drinker who would take an occasional drink in a variety of settings, e.g. as a night cap, during the dinner setting or in a host of other social gatherings. The normal user of alcohol can predict, during social situations, for example, when he or she may imbibe more than they wish to or more than their family may wish them to. The same wish applies during Holiday parties when alcoholic drinks may flow freely. During business luncheons, for example, an individual may want to drink less than normal to stay awake and alert because contracts may have to be negotiated or other decisions must be made. The key in this application is that the population of alcohol users is altogether different from the substance dependent individuals identified in the prior co-pending applications.

As in the co-pending applications, medication sprays are used to accomplish the above noted tasks. any one of several technologies may be used:
1) A nasal spray applicator affecting the vein endings in the mucosal membranes of the nasal passage;
2) A mouth spray affecting the buccal mucosal membrane in the mouth area;
3) A pulmonary inhaler aerosolization into the lungs;
4) Any other delivery system that provides direct absorption through mucus membranes directly into the blood stream.
   For example, the nasal spray may be used by an individual to assist in maintaining control of his or her drinking on a frequent or daily basis because they or their loved ones feel their use of alcohol is excessive. The spray will also act as a behavior trigger to assist in reminding the individual or retraining the individual to mind the frequency, amount or setting of intake of alcohol. In this setting of spray-assisted control of alcohol use there is provided a positive reinforcement of successful moderation, and eventually, the spray assist should no longer be needed.
   From several hours before, and including even while the individual is already drinking, the user of this nasal spray simply squirts one to three doses into each nostril in order to substantially decrease the total consumption of alcohol at any one drinking session. The duration of the effect will vary from individual to individual. The administration of the spray may be repeated as often as one wishes over the period or course of drinking to achieve the desired results, that is, a reduction in the use of alcohol, whether in total amount or in rapidity of alcohol ingestion.
   Case Report:
   A male non-substance dependent individual (M.G.) used this nasal spray by spraying two squirts of the medication “Flumazenil” into each nostril before business dinners on two separate occasions. Without this medication, his usual pattern was the ingestion of one to two martinis before and at the beginning of the meal, followed by one to two glasses of wine during and after the meal.

On each of the two separate occasions where the medication spray was used, M.G. found that his interest in the alcoholic beverages was substantially reduced. His actual intake was less than one martini followed by less than one glass of wine on each of the occasions in question. He also felt that the alcohol had less of an effect on his thinking and feeling than usual on each occasion.

The application or administering of the medicated spray applies to different populations and for different purposes.

1) In the prior applications, an, ill population “abstinent substance dependent individuals”, at risk for relapse, the spray is to promote abstinence and prevent any relapse;

2) In this application, a defined well population, where the aim is to moderate and control the use of alcohol.

A simple analogy would be: The difference between a cruise control on a car versus a governor on a car.

The above discussion honed in on the use of a nasal spray. Of course, the other medication spray technologies mentioned above are equally as applicable. For instance, the mouth spray in the form of Flumazenil is rapidly absorbed by the buccal mucosal membrane in the mouth which will result in a quick absorption into the blood stream of the individual. The third technology is a pulmonary inhaler aerosolization whereby the flumazenil spray is directly absorbed by the tissues in the lungs. Any other delivery system which provides direct absorption through a mucus membrane may also be used.

The aim and purpose of all of the spray technologies discussed above is to administer the medication into the blood stream as quickly as possible with substantially immediate results. It also results in a by-pass of the liver and the intestines, avoiding the so-called “first pass effect” where so much medication is destroyed. Administration of the medication Flumazenil by way of an IV is well known and standard for a variety of medical conditions, previously described, but also recently including detoxification from alcohol or stimulant intoxication. In some settings. While such intravenous use of this medication might accomplish the above noted goals of this application, it is not realistic for an individual to undergo repeated IV therapies on a daily basis, or on an as desired basis, in order to obtain a reduction in alcohol use or to control an intake of alcohol on any given occasion.

Note, that while the use of this spray invention may be used in the actively drinking alcohol dependent individual to attempt to achieve control of alcohol intake on a particular occasion, the primary use for this invention will be in and by the non-dependent individual.

What I claim is:

1. A method for controlling alcohol intake in the non-alcohol dependent individual both before and during his or her imbibing of alcohol, said method comprising the step of an administration to said individual of an effective therapeutic amount of a medication, said administration is undertaken via a spray intake through sensitive mucosal vein endings of said individual on as needed basis, when said individual wants to slow down or reverse the subjective acute alcohol effects he or she is experiencing.

2. The method of claim 1, wherein said step of administering is a self-administering step using a hand held spray device.

3. The method of claim 1, wherein said administering is a wholly new use of said medication named Flumazenil.

4. The method of claim 1, wherein said spray intake is accomplished by way of a nasal spray.

5. The method of claim 1, wherein said spray intake is accomplished by way of a buccal mucosal membrane spray.

6. The method of claim 1, wherein said spray intake is accomplished by way of a pulmonary inhaler aerosolization.

7. A method for attempting to control alcohol intake in the alcohol dependent individual both before and during his or her imbibing of alcohol, said method comprising the step of an administration to said individual of an effective therapeutic amount of a medication, said administration is undertaken via a spray intake through sensitive mucosal vein endings of said individual on as needed basis, when said individual wants to slow down or reverse the subjective acute alcohol effects he or she is experiencing.

8. The method of claim 1, wherein said step of an administration is a self-adminstration step using a hand held spray device.

9. The method of claim 1, wherein said step of administration is a wholly new use of said medication named Flumazenil.

10. The method of claim 1, wherein said spray intake is accomplished by way of a nasal spray.

11. The method of claim 1, wherein said spray intake is accomplished by way of a buccal mucosal membrane spray.

12. The method of claim 1, wherein said spray intake is accomplished by way of a pulmonary inhaler aerosolization.
